(19) United States
(12) Patent Application Publication
(10) Pub. No.: US 2025/0283095 A1
(43) Pub. Date: Sep. 11, 2025

(54) Biradial Spherical Nucleic Acids

(71) Applicant: NORTHWESTERN UNIVERSITY, Evanston, IL (US)

(72) Inventors: Chad A. Mirkin, Wilmette, IL (US); Krishna Sarma Paranandi, Evanston, IL (US)

(21) Appl. No.: 19/073,709

(22) Filed: Mar. 7, 2025

Related U.S. Application Data
(60) Provisional application No. 63/563,079, filed on Mar. 8, 2024.

(51) Int. Cl. C12N 15/115

(52) U.S. Cl. CPC C12N 15/115; C12N 2310/16; C12N 2310/3515; C12N 2310/51

(57) ABSTRACT

The disclosure generally provides biradial spherical nucleic acids (SNAs), compositions comprising a biradial SNA or a plurality thereof, and methods of making and using the biradial SNAs. In some aspects, the disclosure provides a biradial spherical nucleic acid (SNA) comprising: (a) a liposomal core comprising an outer leaflet and an inner leaflet; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, wherein one or more or all of the second plurality of oligonucleotides is a nuclease resistant oligonucleotide. In some embodiments, the first plurality of oligonucleotides and the second plurality of oligonucleotides are different.

### CROSS-REFERENCE TO RELATED APPLICATIONS

This application claims the priority benefit under 35 U.S.C. § 119 (e) of U.S. Provisional Patent Application No. 63/563,079, filed Mar. 8, 2024, which is incorporated herein by reference in its entirety.

### STATEMENT OF GOVERNMENT INTEREST

This invention was made with government support under grant numbers CA257926, CA221747 and CA275430 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2023-205_SeqListing.xml”, which was created on Mar. 4, 2025 and is 12,504 bytes in size. The subject matter of the Sequence Listing is incorporated herein in its entirety by reference.

### BACKGROUND

The intracellular delivery of nucleic acids is a major goal within medicine. This has generally been limited by poor entry of nucleic acids into cells, degradation of nucleic acids by nucleases, and poor control over the types of cells that the oligonucleotides enter. Encapsulating nucleic acids within nanoparticles has been a promising approach for overcoming these challenges, but encapsulation/incorporation efficiencies are often poor owing to repulsion between the negatively charged nucleic acids.

### SUMMARY

As a consequence of the limitations described above, strategies such as the use of cationic/ionizable lipids or polymers are necessary to achieve efficient incorporation, which adds complexity and cost to the resultant system. Spherical nucleic acid (SNA) architectures possess many desired properties (for example and without limitation, enhanced cell entry/multivalent engagement of targets). The present disclosure provides biradial spherical nucleic acids (SNAs) which allow, for example and without limitation, for high encapsulation/incorporation efficiency compared to passive encapsulation without the need for an ionizable lipid and the associated buffer exchanges owing to the presence of the hydrophobic modification, which provides a driving force for internalization. In addition, once the outer oligonucleotides are extracted, a second oligonucleotide can be added on through hydrophobic interactions without the need for click chemistry at this stage, simplifying production.

The biradial spherical nucleic acid is a liposome-based nanostructure comprised of hydrophobically-functionalized oligonucleotides inserted into the inner and outer leaflets of the liposome core. The resultant structure allows for oligonucleotides to be encapsulated into the core with higher efficiency than with passive encapsulation with non-functionalized oligonucleotides. In various embodiments, these oligonucleotides are resistant to the destabilizing influences of serum proteins/nucleases. Using an electrophoresis-based extraction strategy, and in various embodiments, the outer oligonucleotides can be substituted with a different oligonucleotide of distinct sequence/function/labeling. These solvent-exposed oligonucleotides are displayed in a multivalent manner and are capable of binding targets/receptors, enabling enhanced and/or targeted cell uptake through binding to membrane receptors.

Applications of the technology disclosed herein include but are not limited to:
Enhancing intracellular entry of oligonucleotides
Shielding of encapsulated/incorporated oligonucleotides from degradation or the destabilizing influence of serum proteins
Allowing multivalent binding of oligonucleotide binding partners (e.g., scavenger receptors)
Altered cellular handling of differently positioned oligonucleotides

Advantages of the technology disclosed herein include but are not limited to:
Superior encapsulation/incorporation of oligonucleotides (e.g., as compared to passive encapsulation strategies)
Sequence independence (e.g., as compared to hybridization or block co-polymer approaches)
No reliance upon chemical reactions to generate the oligonucleotide-functionalized particles (e.g., as compared to reactions with pendant maleimides or other click chemistry approaches)
No enzymatic degradation required for removal of the outer oligonucleotides, enabling recovery and re-use of extracted oligonucleotides

Accordingly, in some aspects the disclosure provides a biradial spherical nucleic acid (SNA) comprising: (a) a liposomal core comprising an outer leaflet and an inner leaflet; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, wherein one or more or all of the second plurality of oligonucleotides is a nuclease resistant oligonucleotide, and wherein the first plurality of oligonucleotides and the second plurality of oligonucleotides are different. In some embodiments, the liposomal core is comprised of one or more high melting temperature lipids. In further aspects, the disclosure provides a biradial spherical nucleic acid (SNA) comprising: (a) a liposomal core comprising an outer leaflet and an inner leaflet, wherein the liposomal core is comprised of one or more high melting temperature lipids; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, and wherein the first plurality of oligonucleotides and the second plurality of oligonucleotides are different. In some embodiments, the first plurality of oligonucleotides has a surface density of about 0.5 pmol/cm² to about 2 pmol/cm². In further embodiments, the first plurality of oligonucleotides has a surface density of about 0.7 pmol/cm². In some embodiments, the second plurality of oligonucleotides has a surface density of about 0.3 pmol/cm² to about 1.5 pmol/cm². In further embodiments, the second plurality of oligonucleotides has a surface density of about 0.5 pmol/cm². In some embodiments, the liposomal core has a diameter of about 30 nanometers (nm) to about 100 nm. In further embodiments, the liposomal core has a diameter of or of about 60 nm. In still further embodiments, the SNA has a diameter of about 30 nanometers (nm) to about 100 nm. In various embodiments, the one or more high melting temperature lipids is a saturated phosphatidylcholine phospholipid comprising a fatty acid tail length between 16 and 24, a saturated phosphatidylethanolamine lipid with a fatty acid tail length between 16 and 24, a saturated phosphatidylglycerol phospholipid with a fatty acid tail length between 16 and 24, a saturated phosphatidic acid phospholipid with a fatty acid tail length between 16 and 24, a cardiolipin-based phospholipid with a fatty acid tail length between 14 and 16, or a combination thereof. In various embodiments, the liposomal core is comprised of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), or cholesterol. In some embodiments, the liposomal core is comprised of 1,2-dipalmitoyl-sn-glycero-3-phosphocholine (DPPC). In some embodiments, the liposomal core is comprised of one type of lipid. In further embodiments, the liposomal core is comprised of more than one type of lipid. In some embodiments, one or more or all of the first plurality of oligonucleotides is a nuclease resistant oligonucleotide. In further embodiments, one or more or all of the second plurality of oligonucleotides is a nuclease resistant oligonucleotide. In various embodiments, the nuclease resistant oligonucleotide comprises a chemical modification. In some embodiments, one or more oligonucleotides in the population of oligonucleotides comprises a toll-like receptor (TLR) agonist. In some embodiments, each oligonucleotide in the population of oligonucleotides comprises a toll-like receptor (TLR) agonist. In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the TLR agonist is a toll-like receptor 3 (TLR-3) agonist, toll-like receptor 7 (TLR-7) agonist, toll-like receptor 9 (TLR-9) agonist, or a combination thereof. In some embodiments, one or more or all oligonucleotides in the first plurality of oligonucleotides is an aptamer.

In some aspects, the disclosure also provides a method of producing a biradial spherical nucleic acid (SNA), the biradial SNA comprising a liposomal core and a population of oligonucleotides attached to the liposomal core, the method comprising: (a) creating a mixture by contacting the population of oligonucleotides with a population of phospholipids, wherein one or more oligonucleotides in the population of oligonucleotides comprises a hydrophobic anchor group, and wherein the contacting occurs in an aqueous buffer; (b) subjecting the mixture to one or more freeze-thaw cycles; and (c) subjecting the mixture to a process by which diameter of the biradial SNA is reduced, wherein steps (a)-(c) result in a first plurality of oligonucleotides from the population of oligonucleotides being attached to the outer leaflet of the liposomal core and radiating outward from the liposomal core; and a second plurality of oligonucleotides from the population of oligonucleotides being attached to the inner leaflet of the liposomal core and radiating inward into the liposomal core, thereby producing the biradial SNA. In some embodiments, the process is a sonication process. In some embodiments, the process is an extrusion process. In some embodiments, each oligonucleotide in the first plurality of oligonucleotides is identical. In further embodiments, each oligonucleotide in the second plurality of oligonucleotides is identical. In some embodiments, the first plurality of oligonucleotides is identical to the second plurality of oligonucleotides. In some embodiments, each oligonucleotide in the population of oligonucleotides comprises a hydrophobic anchor group. In some embodiments, a method of the disclosure further comprises placing the biradial SNA in a matrix and subjecting the biradial SNA to an electric field, thereby extracting the first plurality of oligonucleotides from the outer leaflet resulting in a liposome comprising the second plurality of oligonucleotides attached to the inner leaflet of the liposomal core. In some embodiments, a method of the disclosure further comprises contacting the liposome comprising the second plurality of oligonucleotides attached to the inner leaflet of the liposomal core with a third plurality of oligonucleotides, wherein each oligonucleotide in the third plurality of oligonucleotides comprises a hydrophobic anchor group, the contacting resulting in the third plurality of oligonucleotides becoming attached to the outer leaflet of the liposomal core, thereby producing a hetero-biradial spherical nucleic acid (SNA). In some embodiments, each oligonucleotide in the third plurality of oligonucleotides is identical. In some embodiments, the second plurality of oligonucleotides are different than the third plurality of oligonucleotides. In some embodiments, one or more oligonucleotides in the population of oligonucleotides comprises a toll-like receptor (TLR) agonist. In some embodiments, each oligonucleotide in the population of oligonucleotides comprises a toll-like receptor (TLR) agonist. In various embodiments, one or more oligonucleotides in the third plurality of oligonucleotides comprises a toll-like receptor (TLR) agonist. In some embodiments, each oligonucleotide in the third plurality of oligonucleotides comprises a toll-like receptor (TLR) agonist. In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the TLR agonist is a toll-like receptor 3 (TLR-3) agonist, toll-like receptor 7 (TLR-7) agonist, toll-like receptor 9 (TLR-9) agonist, or a combination thereof. In some embodiments, one or more or all oligonucleotides in the first plurality of oligonucleotides is an aptamer.

### BRIEF DESCRIPTION OF THE DRAWINGS

FIG. 1 shows that altering the timing of DNA addition generates a biradial SNA structure.

FIG. 2 shows that agarose gel extraction enabled generation of the hetero-biradial SNA structure.

FIG. 3 shows that hetero-biradial TfR Aptamer/T20 SNAs enabled superior T20 oligonucleotide entry into bEND3 cells.

### DETAILED DESCRIPTION

The biradial spherical nucleic acid, owing to its ability to encapsulate oligonucleotides with relatively high efficiency and simultaneously leverage emergent properties that arise from the dense outer arrangement of oligonucleotides, can be used for the transport and delivery of multiple types of oligonucleotide with distinct function and serum sensitivity. The biradial SNA accelerates the material preparation workflow compared to LNP-based platforms by requiring only one lipid component as opposed to the complex lipid mixture found in LNPs while also not requiring the pH alteration and buffer exchange that is needed for loading nucleic acids into ionizable LNPs. Additionally, owing to their endosomal localization, biradial SNAs are uniquely positioned for the modulation of endosomal receptors such as, without limitation, TLR3, 7, and 9.

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 10 percent (%), for example, within 5 percent, 4 percent, 3 percent, 2 percent, or 1 percent of the stated value or range of values.

The terms “polynucleotide” and “oligonucleotide” are interchangeable as used herein.

As used herein, a “hetero-biradial” SNA is one in which the plurality of oligonucleotides attached to the outer leaflet of a liposomal core is different (with respect to, e.g., nucleotide sequence, resistance to nucleases) than the plurality of oligonucleotides attached to the inner leaflet of a liposomal core. Similarly, a “homo-biradial” SNA is one in which the plurality of oligonucleotides attached to the outer leaflet of a liposomal core is the same (with respect to, e.g., nucleotide sequence, resistance to nucleases) as the plurality of oligonucleotides attached to the inner leaflet of a liposomal core.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting, for example, a biradial SNA, a composition comprising a biradial SNA, a therapeutic agent, or a combination thereof to a subject in need of treatment with such an agent. Such modes include, but are not limited to, intravenous, intraarterial, intraperitoneal, intranasal, intrathecal, and subcutaneous administration.

As used herein, “treating” and “treatment” refers to any reduction in the severity and/or onset of symptoms associated with a disorder as disclosed herein. Accordingly, “treating” and “treatment” includes therapeutic and prophylactic measures. One of ordinary skill in the art will appreciate that any degree of protection from, or amelioration of, a disorder is beneficial to a subject, such as a human patient. The quality of life of a patient is improved by reducing to any degree the severity of symptoms in a subject and/or delaying the appearance of symptoms.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. In any of the aspects or embodiments of the disclosure, the immunostimulatory oligonucleotide is a toll-like receptor (TLR) agonist (e.g., a toll-like receptor 9 (TLR9) agonist). In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof.

As used herein, an “immunosuppressive oligonucleotide” is an oligonucleotide that can suppress (e.g., reduce or inhibit) an immune response. Typical examples of immunosuppressive oligonucleotides are TLR antagonists.

All references, patents, and patent applications disclosed herein are incorporated by reference with respect to the subject matter for which each is cited, which in some cases may encompass the entirety of the document.

### Biradial Spherical Nucleic Acids (SNAs)

The disclosure provides biradial spherical nucleic acids (SNAs), compositions comprising a biradial SNA or a plurality thereof, and methods of their use. In some aspects, the disclosure provides a biradial spherical nucleic acid (SNA) comprising: (a) a liposomal core comprising an outer leaflet and an inner leaflet; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, wherein one or more or all of the second plurality of oligonucleotides is a nuclease resistant oligonucleotide, and wherein the first plurality of oligonucleotides and the second plurality of oligonucleotides are different. A liposomal core of the disclosure comprises a lipid bilayer that encloses an aqueous compartment. A “lipid bilayer” generally comprises two monolayers; an outer monolayer referred to herein as an “outer leaflet” and an inner monolayer referred to herein as an “inner leaflet”. A liposomal core is comprised of one or more lipids. As used herein, a “lipid” is a molecule that has hydrophobic or amphiphilic properties. In some embodiments, the amphiphilic properties of some lipids lead them to form a liposomal core in aqueous media. Lipids may be naturally occurring or synthetic. In some embodiments, a lipid of the disclosure is a phospholipid. As used herein, a “phospholipid” is a lipid that comprises a phosphate group. The lipid component of a SNA may include one or more phospholipids, such as one or more (poly) unsaturated lipids. In general, phospholipids may include a phospholipid moiety and one or more fatty acid moieties. In various embodiments, the phospholipid moiety may be one or more of phosphatidyl choline, phosphatidyl ethanolamine, phosphatidyl glycerol, phosphatidyl serine, phosphatidic acid, or a sphingomyelin. In some embodiments, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a liposomal core to facilitate membrane adsorption or in conjugating a liposomal core to one or more oligonucleotides as described herein. In various embodiments, the liposomal core is comprised of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), or cholesterol. In some embodiments, the liposomal core is comprised of 1,2-dipalmitoyl-sn-glycero-3-phosphocholine (DPPC). In some embodiments, the liposomal core is comprised of one or more high melting temperature lipids. A “high melting temperature lipid” is a lipid that is characterized by being more rigid at room and body temperature (e.g., about 25°-37° C.) than an unsaturated lipid (e.g., DOPC). Examples of high melting temperature lipids include, but are not limited to, all saturated phosphatidylcholine phospholipids with a fatty acid tail length between 16 and 24 (e.g., 16:0 PC (DPPC), 17:0 PC, 18:0 PC (DSPC), 19:0 PC, etc.), all saturated phosphatidylethanolamine lipids with a fatty acid tail length between 16 and 24 (e.g., 16:0 PE (DPPE), 17:0 PE, etc.), all saturated phosphatidylglycerol phospholipids with a fatty acid tail length between 16 and 24, all saturated phosphatidic acid phospholipids with a fatty acid tail length between 16 and 24, cardiolipin-based phospholipids with a fatty acid tail length between 14 and 16, and combinations thereof. A “nuclease resistant” oligonucleotide is an oligonucleotide that is modified to increase its resistance to a nuclease relative to an oligonucleotide that is not modified. Chemical modifications contemplated by the disclosure include but are not limited to phosphorothioate linkages, structures such as locked nucleic acids that sterically hinder against nuclease degradation, cationic oligonucleotides (such as DNG oligonucleotides) which are nuclease resistant and have other favorable properties, such as the ability to permeate through cell membranes, or a combination thereof. In some embodiments, only some of the internucleotide linkages in a given oligonucleotide are modified to increase nuclease resistance. In such cases, additional modifications contemplated by the disclosure include but are not limited to those that remove the charge present within the oligonucleotide (such as in peptide nucleic acids or in morpholinos).

In further aspects, the disclosure provides a biradial spherical nucleic acid (SNA) comprising: (a) a liposomal core comprising an outer leaflet and an inner leaflet, wherein the liposomal core is comprised of one or more high melting temperature lipids; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, and wherein the first plurality of oligonucleotides and the second plurality of oligonucleotides are different.

In some embodiments, a biradial SNA of the disclosure has a diameter of, or a population or plurality of biradial SNAs disclosed herein has a mean diameter of, about 30 to about 100 nm. In some embodiments, the mean diameter of the population of biradial SNAs is from about 35 nm to about 100 nm, about 40 nm to about 100 nm, about 45 nm to about 100 nm, about 50 nm to about 80 nm, about 30 nm to about 50 nm, about 30 nm to about 70 nm, about 30 nm to about 60 nm, about 35 nm to about 70 nm, about 40 nm to about 70 nm, or about 30 nm to about 80 nm. In some embodiments, the biradial SNA disclosed herein has a diameter of, or a population or plurality of biradial SNAs disclosed herein has a mean diameter of, about 30 nm to about 90 nm. In some embodiments, the biradial SNA disclosed herein has a diameter of, or a population or plurality of biradial SNAs disclosed herein has a mean diameter of about 60 nm. In some embodiments, the biradial SNA of the disclosure has a diameter of, or a population or plurality of biradial SNAs disclosed herein has a mean diameter of about 30 nm, about 40 nm, about 50 nm, about 60 nm, about 70 nm, about 80 nm, about 90 nm, or about 100 nm. In some embodiments, the biradial SNA of the disclosure has a diameter of, or a population or plurality of biradial SNAs disclosed herein has a mean diameter of about or less than about 30 nm, 35 nm, 40 nm, 50 nm, 60 nm, 70 nm, 80 nm, 90 nm, 100 nm, or any range or combination thereof. In some embodiments, a biradial SNA of the disclosure has a diameter of, or a population or plurality of biradial SNAs disclosed herein has a mean diameter of about or less than 60 nm.

In some embodiments, the liposomal core of a biradial SNA disclosed herein has a diameter of, or the liposomal cores of a population of biradial SNAs disclosed herein has a mean diameter of, about 30 nanometers (nm) to about 100 nm. In some embodiments, the diameter of the liposomal core or the mean diameter of the population of liposomal cores is from about 35 nm to about 100 nm, about 40 nm to about 100 nm, about 45 nm to about 100 nm, about 50 nm to about 80 nm, about 30 nm to about 50 nm, about 30 nm to about 70 nm, about 30 nm to about 60 nm, about 35 nm to about 70 nm, about 40 nm to about 70 nm, or about 30 nm to about 80 nm. In some embodiments, the liposomal core of a biradial SNA disclosed herein has a diameter of, or the liposomal cores of a population of biradial SNAs disclosed herein has a mean diameter of about 30 nm to about 90 nm. In some embodiments, the liposomal core of a biradial SNA disclosed herein has a diameter of, or the liposomal cores of a population of biradial SNAs disclosed herein has a mean diameter of about 60 nm. In some embodiments, the liposomal core of a biradial SNA disclosed herein has a diameter of, or the liposomal cores of a population of biradial SNAs disclosed herein has a mean diameter of about 30 nm, about 40 nm, about 50 nm, about 60 nm, about 70 nm, about 80 nm, about 90 nm, or about 100 nm. In some embodiments, the liposomal core of a biradial SNA disclosed herein has a diameter, or the liposomal cores of a population of biradial SNAs disclosed herein has a mean diameter of about or less than about 30 nm, 35 nm, 40 nm, 50 nm, 60 nm, 70 nm, 80 nm, 90 nm, 100 nm, or any range or combination thereof. In some embodiments, the liposomal core of a biradial SNA disclosed herein has a diameter, or the liposomal cores of a population of biradial SNAs disclosed herein has a mean diameter of about or less than 60 nm.

### Biradial SNA Synthesis

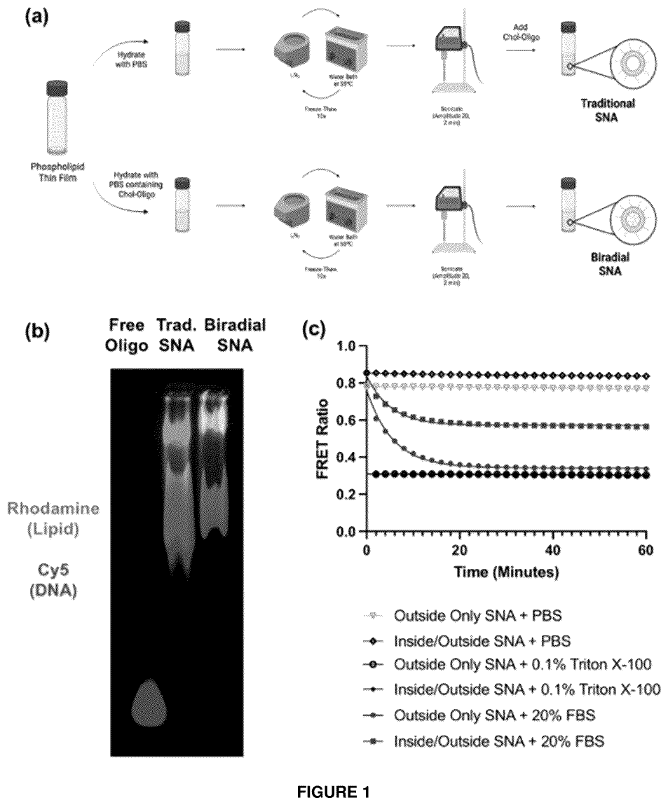

In the conventional process of synthesizing liposomal spherical nucleic acids (LSNAs), small unilamellar vesicles (SUVs) are prepared in a process that involves first hydrating a phospholipid thin film with an aqueous buffer and then subjecting the mixture to freeze-thaw cycles and a down-sizing process, such as sonication or extrusion. Oligonucleotides modified with a hydrophobic anchor group are then added into the SUV mixture, generating LSNAs in which oligonucleotides are radially oriented on the outer leaflet of the liposome. In biradial SNA synthesis, and in some non-limiting exemplary methods, the process is modified such that, for example, the hydrophobically-functionalized oligonucleotides are mixed into the aqueous buffer that is used to hydrate the thin film. The resultant mixture is then freeze-thawed and sonicated/extruded in the conventional manner. In this way, oligonucleotides have equal access to both leaflets of the liposomal core while the liposomes are forming and while they are broken apart and reformed during the freeze-thaw cycles and sonication/extrusion, thus allowing insertion of the oligonucleotides into both the inner and outer leaflets of the liposomal core, generating a “homo-biradial SNA”. Through use of agarose gel electrophoresis and FRET serum stability assays on fluorophore-labeled SNAs (FIG. 1), this may be confirmed.

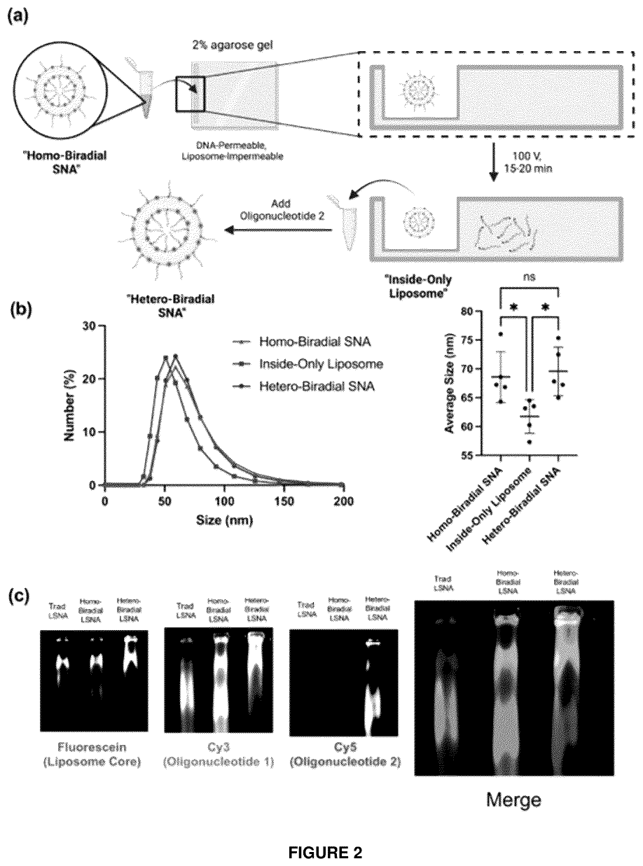

In order to prepare hetero-biradial SNAs, in which different oligonucleotides are positioned on the outer and inner leaflets, homo-biradial SNAs are first prepared as described above. These structures are then loaded into a 2% agarose gel, which contains pores that are large enough to permit passage of oligonucleotides but not of the liposomal cores. The electric field is then turned on (typical parameters—100 V, 15-20 mins), resulting in the outer oligonucleotides being extracted into the gel and “inside-only liposomes” being left behind in the well. These liposomes can then be extracted from the well and functionalized with a second hydrophobically-functionalized oligonucleotide, thereby generating the hetero-biradial SNA. In this way, biradial SNAs can be prepared such that the inner leaflet and outer leaflet oligonucleotides can be imparted with distinct functions. This process has been validated through dynamic light scattering analysis of the homo-biradial, inside-only liposome, and hetero-biradial structures as well as through agarose gel electrophoresis of fluorophore-labeled SNAs (FIG. 2). By “different” oligonucleotides it is meant that the oligonucleotides on the outer leaflet are structurally distinct from the oligonucleotides on the inner leaflet. Structurally distinct features include, without limitation, different nucleotide sequences that may impart distinct functionality (e.g., the first plurality of oligonucleotides may be immunostimulatory oligonucleotides while the second plurality of oligonucleotides may be a homopolymeric (e.g., polyG) nucleotide sequence), different oligonucleotide backbones (e.g., the first plurality of oligonucleotides may comprise a phosphorothioate backbone while the second plurality of oligonucleotides does not comprise a phosphorothioate backbone), nuclease resistance (e.g., the first plurality of oligonucleotides may be designed to be nuclease resistant as described herein while the second plurality of oligonucleotides is not designed to be nuclease resistant), or a combination thereof. Similarly, by “identical” oligonucleotides it is meant that the oligonucleotides on the outer leaflet are structurally identical to the oligonucleotides on the inner leaflet. Structurally distinct features are as described above and include, without limitation, nucleotide sequences that may impart functionality (e.g., both the first plurality of oligonucleotides and the second plurality of oligonucleotides are immunostimulatory oligonucleotides, both the first plurality of oligonucleotides and the second plurality of oligonucleotides are homopolymeric (e.g., polyG) nucleotide sequences), same oligonucleotide backbones (e.g., both the first plurality of oligonucleotides and the second plurality of oligonucleotides comprise a phosphorothioate backbone), nuclease resistance (e.g., both the first plurality of oligonucleotides and the second plurality of oligonucleotides are designed to be nuclease resistant as described herein), or a combination thereof. It will be appreciated that combinations of the foregoing are also contemplated. Thus, in some embodiments, each oligonucleotide in the first plurality of oligonucleotides is identical. In further embodiments, each oligonucleotide in the second plurality of oligonucleotides is identical. In still further embodiments, the first plurality of oligonucleotides is identical to the second plurality of oligonucleotides. In some embodiments, each oligonucleotide in the second plurality of oligonucleotides is identical, each oligonucleotide in the second plurality of oligonucleotides is identical, and the first plurality of oligonucleotides and the second plurality of oligonucleotides are different.

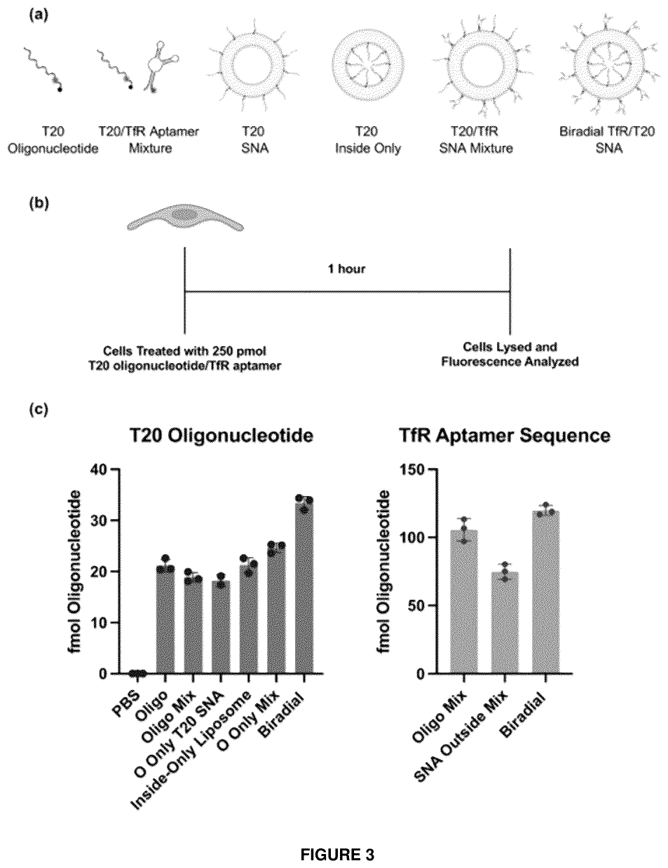

In some embodiments, the disclosure provides a hetero-biradial SNA wherein the oligonucleotides on the outer leaflet are designed to maximize cell entry, while the oligonucleotides on the inner leaflet comprise a distinct sequence of choice. To illustrate this, hetero-biradial SNAs were prepared featuring aptamers to transferrin receptors, which are abundant on brain endothelial cells, on the outside and an arbitrary T20 oligonucleotide on the inside. These structures allowed superior internalization of T20 oligonucleotides into brain endothelial cells compared to other structures/controls (FIG. 3).

### Oligonucleotides

The disclosure provides biradial spherical nucleic acids (SNAs) comprising: (a) a liposomal core comprising an outer leaflet and an inner leaflet; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, wherein one or more or all of the second plurality of oligonucleotides is a nuclease resistant oligonucleotide, and wherein the first plurality of oligonucleotides and the second plurality of oligonucleotides are different. In some aspects, the disclosure provides a biradial spherical nucleic acid (SNA) comprising: (a) a liposomal core comprising an outer leaflet and an inner leaflet, wherein the liposomal core is comprised of one or more high melting temperature lipids; and (b) a population of oligonucleotides attached to the liposomal core, the population of oligonucleotides comprising: (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core, and wherein the first plurality of oligonucleotides and the second plurality of oligonucleotides are different.

Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate or phosphodiester linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures, for example as described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH=(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH=(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which-CH₂—CO—NR^{H}_, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzox-azin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 5 nucleotides to about 100 nucleotides in length. More specifically, an oligonucleotide of the disclosure is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, about 15 to about 27 nucleotides in length, about 15 to about 28 nucleotides in length, about 15 to about 29 nucleotides in length, about 15 to about 30 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In some embodiments, an oligonucleotide of the disclosure is or is about 25 nucleotides in length. In various embodiments, the population of oligonucleotides attached to the liposomal core of the biradial SNA comprises a plurality of oligonucleotides that all have the same length/sequence, while in some embodiments, the plurality of oligonucleotides comprises one or more oligonucleotides that have a different length and/or sequence relative to at least one other oligonucleotide in the plurality.

In various embodiments, one or more or all of the oligonucleotides in the population of oligonucleotides attached to the liposomal core is an aptamer. In some embodiments, one or more or all oligonucleotides in the first plurality of oligonucleotides is an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Spacers. In some aspects and embodiments, one or more or all oligonucleotides in the population of oligonucleotides that is attached to the liposomal core of a biradial SNA comprise a spacer. “Spacer” as used herein means a moiety that serves to increase distance between the liposomal core and the oligonucleotide, or to increase distance between individual oligonucleotides when attached to the liposomal core in multiple copies, or to improve the synthesis of the biradial SNA. Thus, spacers are contemplated being located between an oligonucleotide and the liposomal core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the liposomal core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

Biradial SNA density. The disclosure generally provides biradial SNAs comprising a population of oligonucleotides attached to the liposomal core, the population of oligonucleotides comprising (i) a first plurality of oligonucleotides attached to the outer leaflet of the liposomal core and radiating outward from the liposomal core; and (ii) a second plurality of oligonucleotides attached to the inner leaflet of the liposomal core and radiating inward into the liposomal core. In various embodiments, the disclosure contemplates that the first plurality of oligonucleotides attached to the outer leaflet of the liposomal core has a density of oligonucleotides that is about 0.5 pmol/cm² to about 2 pmol/cm². In some embodiments, the first plurality of oligonucleotides attached to the outer leaflet of the liposomal core has a density of oligonucleotides that is, is about, or is at least about 0.7 pmol/cm². In further embodiments, the first plurality of oligonucleotides attached to the outer leaflet of the liposomal core has a density of oligonucleotides that is about 0.7 pmol/cm² to about 2 pmol/cm², about 0.9 pmol/cm² to about 2 pmol/cm², about 1.0 pmol/cm² to about 2 pmol/cm², about 1.2 pmol/cm² to about 2 pmol/cm², about 1.5 pmol/cm² to about 2 pmol/cm², about 1.7 pmol/cm² to about 2 pmol/cm², about 0.7 pmol/cm² to about 1.5 pmol/cm², about 0.7 pmol/cm² to about 1.2 pmol/cm², about 0.7 pmol/cm² to about 1.0 pmol/cm², about 0.5 pmol/cm² to about 1.5 pmol/cm², about 0.5 pmol/cm² to about 1.0 pmol/cm², about 0.5 pmol/cm² to about 1.0 pmol/cm², or about 0.5 pmol/cm² to about 0.8 pmol/cm². In still further embodiments, the first plurality of oligonucleotides attached to the outer leaflet of the liposomal core has a density of oligonucleotides that is, is about, is at least about, or is less than about 0.5 pmol/cm², 0.7 pmol/cm², 0.9 pmol/cm², 1.1 pmol/cm², 1.3 pmol/cm², 1.5 pmol/cm², 1.7 pmol/cm², 1.9 pmol/cm², or 2.0 pmol/cm². In some embodiments, the disclosure contemplates that the second plurality of oligonucleotides attached to the inner leaflet of the liposomal core has a density of oligonucleotides that is about 0.3 pmol/cm² to about 1.5 pmol/cm². In some embodiments, the disclosure contemplates that the second plurality of oligonucleotides attached to the inner leaflet of the liposomal core has a density of oligonucleotides that is, is about, or is at least about 0.5 pmol/cm². In further embodiments, the second plurality of oligonucleotides attached to the inner leaflet of the liposomal core has a density of oligonucleotides that is about 0.3 pmol/cm² to about 1.5 pmol/cm², about 0.5 pmol/cm² to about 1.0 pmol/cm², about 0.7 pmol/cm² to about 0.9 pmol/cm², about 0.7 pmol/cm² to about 1.5 pmol/cm², about 0.9 pmol/cm² to about 1.5 pmol/cm², about 1.0 pmol/cm² to about 1.5 pmol/cm², about 1.2 pmol/cm² to about 1.5 pmol/cm², about 0.7 pmol/cm² to about 1.2 pmol/cm², about 0.7 pmol/cm² to about 1.0 pmol/cm², about 0.5 pmol/cm² to about 1.5 pmol/cm², about 0.5 pmol/cm² to about 1.0 pmol/cm², about 0.5 pmol/cm² to about 1.3 pmol/cm², or about 0.5 pmol/cm² to about 0.8 pmol/cm². In still further embodiments, the second plurality of oligonucleotides attached to the inner leaflet of the liposomal core has a density of oligonucleotides that is, is about, is at least about, or is less than about 0.3 pmol/cm², 0.5 pmol/cm², 0.7 pmol/cm², 0.9 pmol/cm², 1.1 pmol/cm², 1.3 pmol/cm², or 1.5 pmol/cm².

Alternatively, the density of oligonucleotide attached to the biradial SNA is measured by the number of oligonucleotides attached to the biradial SNA. With respect to the density of oligonucleotides attached to a biradial SNA of the disclosure, it is contemplated that, in various embodiments, a biradial SNA comprises about 1 to about 150, about 1 to about 120, about 1 to about 100, about 1 to about 90, about 1 to about 80, about 1 to about 75, about 1 to about 70, about 1 to about 60, about 1 to about 50, about 1 to about 40, about 1 to about 30, about 1 to about 20, or about 1 to about 10 oligonucleotides in total (e.g., the total number of oligonucleotides in the first plurality of oligonucleotides and the second plurality of oligonucleotides). In various embodiments, the first plurality of oligonucleotides attached to the outer leaflet of the liposomal core of a biradial SNA as described herein comprises about 1 to about 80 oligonucleotides. In further embodiments, the first plurality of oligonucleotides attached to the outer leaflet of the liposomal core of a biradial SNA as described herein comprises about 1 to about 75, about 1 to about 70, or about 1 to about 65, or about 1 to about 60, or about 1 to about 55, about 1 to about 50, or about 1 to about 45, or about 1 to about 40, or about 1 to about 35, or about 1 to about 30, or about 1 to about 25, or about 1 to about 20, or about 1 to about 15, or about 1 to about 10, or about 1 to about 5, or about 10 to about 80, or about 10 to about 70, or about 10 to about 60, or about 10 to about 50, or about 10 to about 45, or about 10 to about 40, or about 10 to about 35, or 10 to about 30, or about 10 to about 25, or about 10 to about 20, or about 10 to about 15 oligonucleotides. In further embodiments, the first plurality of oligonucleotides attached to the outer leaflet of the liposomal core of a biradial SNA as described herein comprises about or at least about 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 oligonucleotides. In further embodiments, the first plurality of oligonucleotides attached to the outer leaflet of the liposomal core of a biradial SNA as described herein comprises less than about 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 oligonucleotides. In still further embodiments, the first plurality of oligonucleotides attached to the outer leaflet of the liposomal core of a biradial SNA as described herein consists of 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or 80 oligonucleotides. In further embodiments, it is contemplated that the second plurality of oligonucleotides attached to the inner leaflet of the liposomal core of a biradial SNA as described herein comprises about 1 to about 40 oligonucleotides. In various embodiments, the second plurality of oligonucleotides attached to the inner leaflet of the liposomal core of a biradial SNA as described herein comprises about 1 to about 30, or about 1 to about 25, or about 1 to about 20, or about 1 to about 15, or about 1 to about 10, or about 1 to about 5, or about 10 to about 40, or about 10 to about 35, or 10 to about 30, or about 10 to about 25, or about 10 to about 20, or about 10 to about 15 oligonucleotides. In further embodiments, the second plurality of oligonucleotides attached to the inner leaflet of the liposomal core of a biradial SNA as described herein comprises about or at least about 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, or 40 oligonucleotides. In further embodiments, the second plurality of oligonucleotides attached to the inner leaflet of the liposomal core of a biradial SNA as described herein comprises less than about 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, or 40 oligonucleotides. In still further embodiments, the second plurality of oligonucleotides attached to the inner leaflet of the liposomal core of a biradial SNA as described herein consists of 1, 2, 3, 4, 5, 6, 7, 8, 9, 10, 11, 12, 13, 14, 15, 16, 17 18, 19, 20, 21, 22, 23, 24, 25, 26, 27, 28, 29, 30, 31, 32, 33, 34, 35, 36, 37, 38, 39, or 40 oligonucleotides.

### Compositions

The disclosure also provides compositions that comprise a biradial SNA of the disclosure, or a plurality thereof. In some embodiments, the composition further comprises a pharmaceutically acceptable carrier. The term “carrier” refers to a vehicle within which the biradial SNA as described herein is administered to a subject. Any conventional media or agent that is compatible with the biradial SNAs according to the disclosure can be used. The term carrier encompasses diluents, excipients, adjuvants and a combination thereof. Pharmaceutically acceptable carriers are well known in the art (see, e.g., Remington's Pharmaceutical Sciences by Martin, 1975, the entire disclosure of which is herein incorporated by reference).

Exemplary “diluents” include water for injection, saline solution, buffers such as Tris, acetates, citrates or phosphates, fixed oils, polyethylene glycols, glycerine, propylene glycol or other synthetic solvents. Exemplary “excipients” include but are not limited to stabilizers such as amino acids and amino acid derivatives, polyethylene glycols and polyethylene glycol derivatives, polyols, acids, amines, polysaccharides or polysaccharide derivatives, salts, and surfactants; and pH-adjusting agents.

### Uses of Biradial SNAS to Treat a Disorder

In some embodiments, a biradial SNA of the disclosure is used to treat a disorder. Thus, in some aspects, the disclosure provides methods of treating a disorder comprising administering an effective amount of a biradial SNA or composition of the disclosure to a subject (e.g., a human subject) in need thereof, wherein the administering treats the disorder. In various embodiments, the disorder is cancer, a neurological disorder, a cardiovascular disease, a liver disorder, a kidney disease, or a combination thereof. An “effective amount” of the biradial SNA is an amount sufficient to, for example, treat, ameliorate, and/or prevent the disorder.

A biradial SNA of the disclosure can be administered via any suitable route, for example and without limitation parenteral administration, intravenous, intraarterial, intraperitoneal, intranasal, intrathecal, and/or subcutaneous administration. A combination of different routes of administration, separately or at the same time, is also contemplated by the disclosure.

### Therapeutic Agents

In some embodiments, the biradial SNAs or compositions comprising a biradial SNA provided herein further comprise a therapeutic agent, or a plurality thereof. In some embodiments, the therapeutic agent is attached directly to the liposomal core of the biradial SNA. In some embodiments, the disclosure provides biradial SNAs wherein one or more therapeutic agents are covalently and/or non-covalently associated with oligonucleotides in the shell of oligonucleotides that is attached to the exterior of the liposomal core of the biradial SNA. It will also be understood that non-covalent associations include hybridization, protein binding, and/or hydrophobic interactions. In some embodiments, a therapeutic agent is administered separately from a biradial SNA of the disclosure. Thus, in some embodiments, a therapeutic agent is administered before, after, or concurrently with a biradial SNA of the disclosure to treat a disorder.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### EXAMPLES

The biradial spherical nucleic acid is a type of spherical nucleic acid architecture characterized by oligonucleotides positioned on the inner and outer leaflets of a liposome core. In the homo-biradial structure, the same nucleic acid is used on both leaflets. In the hetero-biradial structure, distinct oligonucleotides are used on the inner and outer leaflets.

### Example 1

### Experimental Section

Oligonucleotide Synthesis and Characterization. Oligonucleotide sequences were synthesized using phosphoramidite coupling chemistry on an automated DNA synthesizer (Applied Biosystems), purified using high-performance liquid chromatography, and characterized using matrix-assisted laser desorption/ionization-time of flight (MALDI-TOF) mass spectrometry (Bruker) as previously described. All sequences had the design shown below:
5′-Cholesterol-spacer18-Fluorophore-spacer18-sequence-3′

### Sequences Used:

Cy5 Labeled
(SEQ ID NO: 1)
Cholesterol-spacer18-Cy5-spacer18-TCC ATG ACG TTC
CTG ACG TT
(SEQ ID NO: 2)
Cholesterol-spacer18-Cy5-spacer18-TTT TTT TTT TTT
TTT TTT TT
(SEQ ID NO: 3)
Cholesterol-spacer18-Cy5-spacer18-TCC CAG TGC CCA
GTC CAA C
Cy3 Labeled
(SEQ ID NO: 4)
Cholesterol-spacer18-Cy3-spacer18- CCT GGA TGG GAA
CTT ACC GCT GCA
(SEQ ID NO: 5)
Cholesterol-spacer18-Cy3-spacer18-CCT TCC CTG AAG
GTT CCT CC
(SEQ ID NO: 6)
Cholesterol-spacer18-Cy3-spacer18- GCG TGG TAC CAC
GCT TTT T

Traditional and Biradial SNA Preparation and Characterization. To prepare SNAs, a phospholipid thin film comprised of DOPC or DPPC and rhodamine-labeled phospholipid (Avanti Polar Lipids) was first prepared by evaporating chloroform solutions containing approximately 3000 nmol of unlabeled lipid and 30 nmol of labeled lipid in a glass vial. For traditional SNAs, this solution was then hydrated with phosphate-buffered saline (PBS), while for biradial SNAs, the solution was hydrated with a PBS solution containing approximately 6 nmol of cholesterol-functionalized, Cy5-labeled oligonucleotide (1826 sequence (SEQ ID NO: 1)). The hydrated solution was then subject to ten freeze-thaw cycles and then sonicated (amplitude 20, 3 sec pulses, 5 sec rest, pulse time 2 min) using a probe-tip sonicator (Fisher Scientific). For traditional SNAs, 6 nmol of the oligonucleotide was then added, and both solutions were allowed to incubate at 55° C. overnight. To assess for SNA formation, linear oligonucleotide, traditional SNA, and biradial SNA were added into a 1% agarose gel and run at 120 V for 1 h. The gel was then examined with a ChemiDoc MP fluorescence imager (Bio-Rad).

FRET Serum Stability Assay. To analyze the serum stability of traditional and biradial SNAs, 100 μL of traditional or biradial SNA solutions containing approximately 60 pmol of DNA and 300 pmol of rhodamine-functionalized lipid were added to into a 96-well plate. To these wells, either PBS, fetal bovine serum to a final concentration of 20%, or Triton X-100 to a final concentration of 0.1% were added such that the final solution volume was 200 μL. Rhodamine-Cy5 fluorescence resonance energy transfer (FRET) was measured every 2 min for 60 min by measuring rhodamine donor fluorescence at excitation/emission wavelengths of 550/585 nm and Cy5 acceptor fluorescence at excitation/emission wavelengths of 550/670 nm. The FRET ratio was then calculated by dividing the acceptor fluorescence by the sum of the acceptor and donor fluorescence.

Agarose Extraction Procedure. To perform the agarose extraction of solvent-exposed oligonucleotides, biradial SNAs with the same oligonucleotide displayed on both leaflets (homo-biradial SNAs) were prepared as described above with the modification of using a 7-nitro-2-1,3-benzoxadiazol-4-yl (NBD)-functionalized phospholipid and a Cy3-functionalized oligonucleotide. These homo-biradial SNAs were then loaded into a 2% agarose gel, and the gel was run in 1×TBE buffer at 100 V for 20 min. After this, the material left behind in the well (inside-only liposomes) was collected, and the amount of DNA was quantified through comparison against a fluorescence standard curve. An equal amount of a cholesterol-functionalized, Cy5-labeled oligonucleotide was then added to the solution of inside-only liposomes in order to generate the hetero-biradial SNAs. These structures were then run through a Sepharose CL-4B size exclusion column to exchange the buffer into PBS. To characterize the structures, the size of the homo-biradial SNAs, inside-only liposomes, and hetero-biradial SNAs was assessed through dynamic light scattering on a Zetasizer instrument (Malvern). In addition, the traditional, homo-biradial, and hetero-biradial SNAs prepared with NBD-functionalized phospholipids, Cy3-labeled oligonucleotides, and Cy5-labeled oligonucleotides were analyzed on a 1% agarose gel in 1×TBE at 120 V for 1 h.

Cell Uptake Analysis. For bEnd3 cell uptake studies, the bEnd.3 cells were seeded into a 48-well plate at a density of 3×10⁴ cells per well in a complete growth medium (Dulbecco's modified Eagle medium, 10% heat-inactivated fetal bovine serum, 1% penicillin/streptomycin) and allowed to attach for 48 hours. The cells were then treated with either PBS, a cholesterol- and Cy5-functionalized T20 oligonucleotide, a mixture of the T20 oligonucleotide and a cholesterol-functionalized, Cy3-labeled aptamer to the transferrin receptor (TfR), a traditional SNA functionalized with the T20 oligonucleotide, an inside-only liposome containing the T20 oligonucleotide, a traditional SNA co-functionalized with the T20 oligonucleotide and TfR aptamer, and a hetero-biradial SNA with the TfR aptamer positioned on the external leaflet and the T20 sequence positioned on the inner leaflet. Treatments were carried out at a T20 oligonucleotide and, for treatments that had it, TfR aptamer concentration of 1 μM (50 μL of a 5 μM solution added into 200 μL of media, 250 pmol of each oligonucleotide). After 1 hour of treatment, the cells were washed with 1×PBS and then lysed with 1×RIPA buffer. Fluorescence was then compared to that of Cy5 and Cy3 standard curves in order to determine the amount of oligonucleotide in the cell homogenate.

### Results

Altering the Timing of DNA Addition Generates a Biradial SNA Architecture. In order to produce a biradial LSNA structure featuring oligonucleotides positioned on both leaflets of the liposome core, the conventional procedure for producing LSNAs was modified by altering the timing of DNA addition (FIG. 1a). In the conventional process of synthesizing LSNAs, small unilamellar vesicles (SUVs) are prepared in a process that involves first hydrating a phospholipid thin film with an aqueous buffer and then subjecting the mixture to freeze-thaw cycles and a down-sizing process, such as sonication or extrusion. Oligonucleotides modified with a hydrophobic moiety, such as cholesterol, are then added into the SUV mixture, generating LSNAs in which oligonucleotides are radially oriented on the outer leaflet of the liposome. For biradial SNA synthesis, the process is modified such that the hydrophobically-functionalized oligonucleotides are mixed into the aqueous buffer that is used to hydrate the thin film. The resultant mixture is then freeze-thawed and down-sized in the conventional manner. In this way, the oligonucleotides have access to both leaflets of the liposome cores as they are forming as well as while the cores are broken apart and reformed during the subsequent workup process, which should allow for insertion into either leaflet. In order to assess for this, traditional and biradial SNAs were prepared using Cy5-labeled oligonucleotides and liposomes containing rhodamine-labeled phospholipids and then analyzed via agarose gel electrophoresis (FIG. 1b). Compared to the linear oligonucleotide, the oligonucleotides in traditional SNAs displayed an upward mobility shift, indicating a mobility reduction owing to initial association with the liposome cores, and complete separation from the rhodamine-labeled core material, reflecting complete separation of the oligonucleotides and core within the gel during the electrophoresis run. Biradial SNAs similarly showed a fraction of oligonucleotide material that separates from the rhodamine core and displays an upward mobility shift relative to the linear oligonucleotide but, in contrast, also showed a fraction of oligonucleotide material that remained colocalized with the rhodamine core, reflecting the presence of an internalized fraction of oligonucleotide.

To further assess the architecture and to determine whether internalizing oligonucleotides within the liposome core protects against serum protein-induced oligonucleotide dissociation, a fluorescence resonance energy transfer (FRET)-based serum stability assay was also performed by incubating traditional and biradial SNAs in 20% fetal bovine serum (FBS) at 37° C. and assessing for the decline in rhodamine-Cy5 FRET (FIG. 1c). Both traditional and biradial SNAs incubated in PBS at 37° C. displayed a constant rhodamine-Cy5 FRET ratio over a 60 min period. When 0.1% Triton X-100, a detergent that destroys the liposomal architecture, was added, the FRET ratio for both structures declined dramatically. When traditional SNAs were incubated in 20% FBS, the FRET ratio exponentially declined from its value in PBS down to the values observed for Triton X-100-treated SNAs, reflecting a complete dissociation of oligonucleotides from the liposome core. On the other hand, when biradial SNAs were incubated in 20% FBS, the FRET ratio exponentially declined to a plateau point between the Triton X-100 minimum and the starting FRET value, reflecting incomplete oligonucleotide separation from the liposome core. This provided further confirmation that oligonucleotides are shielded from solvent exposure in the biradial structure and that this shielding confers resistance against serum protein-triggered dissociation.

Agarose Extraction Allows for Generation of the Hetero-Biradial SNA. Having prepared biradial SNAs with the same oligonucleotide distributed across both liposome leaflets (“homo-biradial SNAs”), it was next sought to develop a method for preparing “hetero-biradial SNAs” with distinct oligonucleotides on the inner and outer leaflets. In order to do this, a homo-biradial SNA structure comprised of an NBD/fluorescein-labeled liposome core and Cy3-labeled oligonucleotides was first prepared. These homo-biradial constructs were then loaded into a preparatory-scale 2% agarose gel (FIG. 2a). This gel permitted the passage of oligonucleotides into the gel matrix but not the larger liposome cores, which remained behind in the well. After running the gel at 100 V for 15-20 min, the material left behind in the well was collected. These structures, so-called “inside-only liposomes” which lack oligonucleotides on the outer leaflet, were then functionalized with a second cholesterol-functionalized, Cy5-labeled oligonucleotide in order to generate the hetero-biradial SNA structure.

In order to validate that oligonucleotides were being extracted while leaving the liposome cores intact, the hydrodynamic diameter of the homo-biradial SNAs and inside-only liposomes were compared using dynamic light scattering (FIG. 2b). This showed that there was a size decline of the inside-only liposomes relative to the homo-biradial SNAs, a finding that is attributable to loss of the outer oligonucleotide shell. When the second oligonucleotide sequence was added to the inside-only liposomes to produce the hetero-biradial SNAs, the size increased back to the original size average, confirming that the nucleic acid shell was restored. To verify the positioning of oligonucleotides within the SNA, traditional, homo-biradial, and hetero-biradial SNAs were run on a 1% analytical agarose gel (FIG. 2c). Similar to the prior observation, the traditional SNA featured complete separation of the oligonucleotides from the core within the gel and the homo-biradial SNA showed an external fraction that separated and an internal fraction that remained associated with the core. The hetero-biradial SNAs showed a core-colocalizing fraction and a core-separating fraction, with the colocalizing, internal fraction comprised of the original Cy3 oligonucleotide and the separating, external fraction comprised of the added Cy5 oligonucleotide, demonstrating that a biradial structure had been developed in which distinct oligonucleotides are present on the inner and outer leaflets.

Biradial TfR/T20 SNAs Enable Superior Entry of T20 Oligonucleotides into bEND.3 Cells. It was next assessed whether the biradial SNA architecture could be leveraged to enhance the uptake of SNAs into cells of interest by decorating the outside of the SNA with a receptor-binding oligonucleotide and incorporating a cargo oligonucleotide within the core of the structure. It has previously been found that aptamers directed against the transferrin receptor (TfR), which is abundantly expressed on brain endothelial cells, can be used to promote the uptake of SNA structures with protein-based cores into the brain endothelium. In order to determine if biradial SNAs could be used in a similar manner to internalize a non-aptamer T20 oligonucleotide sequence into brain endothelial cells, a set of linear and nanoscale structures (FIG. 3a) including a linear T20 oligonucleotide, a simple mixture of a T20 oligonucleotide and TfR aptamer, a traditional SNA with T20 oligonucleotides on the surface, an inside-only liposome with T20 oligonucleotides anchored into the inner leaflet of the liposome, a traditional SNA with T20 oligonucleotides and TfR aptamers dispersed on the surface, and a biradial structure in which TfR aptamers were displayed on the outside and T20 oligonucleotides anchored to the inner leaflet were prepared. These materials were incubated at a T20 oligonucleotide (and, for treatments containing it, TfR aptamer concentration) of 1 μM (250 pmol in 250 μL) with bEnd.3 cells, a commonly used murine brain endothelial cell line, for 1 h, after which the cells were lysed and quantitatively analyzed for cell uptake (FIG. 3b). This analysis showed that, when treatment was done with the T20 oligonucleotide on its own, whether in linear, traditional SNA, or inside-only liposome form, or in simple mixture form with the TfR aptamer, approximately 20 fmol of the T20 oligonucleotide was internalized over the 1 h incubation (FIG. 3c, left). Over the same 1 h interval, approximately 100 fmol (FIG. 3c, right) of the TfR aptamer was internalized when it was used in a simple mixture with the T20 oligonucleotide, reflecting the higher propensity that this sequence has for uptake into these cells. When SNAs containing T20 oligonucleotides and TfR aptamers both dispersed on the surface were used, T20 oligonucleotide uptake modestly increased, but the uptake of the TfR aptamer was reduced, suggesting that the affinity for uptake was reduced when the TfR aptamer is forced onto the same surface as a non-aptameric sequence. On the other hand, when a biradial SNA structure with TfR aptamers positioned on the outside surface and T20 oligonucleotides positioned on the inner leaflet was used, T20 oligonucleotide uptake sharply increased, and TfR aptamer uptake was restored, indicating that this structure is one example of a design for maximizing uptake.

### Example 2

Enhancing the Uptake of Immunostimulatory SNAs into Macrophages/Antigen-Presenting Cells

Design and Expected Results: G-quadruplex-containing DNA on the outside, TLR9 agonist DNA on the inside. This design based on the idea that oligonucleotide sequences containing a G-quadruplex secondary structure are known to better interact with scavenger receptor A, a cell surface receptor abundantly expressed on the surface of macrophages and other antigen-presenting cells, compared to non-structured sequences. Because of this, biradial SNAs having a G-quadruplex rich sequence on the outside and a TLR9-stimulating oligonucleotide on the inside will enable higher internalization of the cargo immunostimulatory oligonucleotide, thus enhancing the immune stimulation that is achieved with a particular amount of immunostimulatory oligonucleotide.

### Methods and Experiments

Hetero-biradial SNAs will be prepared according to the methods described herein (e.g., Example 1) using a TLR9 stimulating oligonucleotide (5′-Cholesterol-sp18-Cy5-sp18-TCC ATG ACG TTC CTG ACG TT-3′ (SEQ ID NO: 1), phosphorothioate (PS) backbone) as the inner oligonucleotide and using a G-rich oligonucleotide sequence capable of forming a G-quadruplex secondary structure as the outer oligonucleotide. The G-quadruplex-rich sequence will have the design of 5′-Cholesterol-sp18-Cy3-sp18-GGT GGT GGT GGT GGT GGT GGT-3′, (SEQ ID NO: 7), phosphodiester (PO) backbone. Formation of the G-quadruplex secondary structure will be confirmed using circular dichroism spectroscopy. To assess for the ability of these hetero-biradial constructs to enhance the uptake and immunostimulatory activity of the TLR9-stimulating cargo oligonucleotide, RAW264.7 macrophages will be pulsed with these SNAs, with hetero-biradial SNAs having the TLR9 stimulating oligonucleotide on the outside and G-rich oligonucleotide on the inside, and with conventional SNAs having TLR9-stimulating oligonucleotides on the outside for 1-2 hours. Then, the cells will be washed, and a subset of cells will be lysed with 1×RIPA buffer, and the lysate harvested. The remaining cells will have their overlying media replaced with fresh media. After 24 hours, the media will be collected. Levels of pro-inflammatory cytokines like TNFα and IL-6 will be assessed in the overlying media, while Cy5 fluorescence will be quantified within the cell lysates. It is expected that cells treated with the hetero-biradial SNA having G-quadruplex oligonucleotides on the outside and TLR9 stimulating oligonucleotides on the inside will display superior internalization of the Cy5-labeled TLR9-stimulating oligonucleotide and will consequently display higher levels of pro-inflammatory cytokines in the overlying media.

If this is not observed, then potential explanations are that the G-quadruplex oligonucleotide is dissociating from the LSNA surface prior to cell entry and/or that the internal TLR9-stimulating oligonucleotide is not able to access the TLR9 receptor. To assess for the former, the G-quadruplex sequence will be anchored into the outer membrane using a phospholipid anchor, which is less prone to dissociation than a cholesterol anchor. To assess for the latter, the liposome core composition will be modified by incorporating 20-30% N-palmitoyl homocysteine (PHC) lipid into the liposome. PHC is a pH-sensitive lipid that leads to liposome structure breakdown upon acidification, particularly in the pH range that is observed in the early endosome. As a consequence, PHC-containing liposomes tend to release their cargo within the early endosome, where it may then interact with TLR9.

Shielding Charged Nucleic Acids from Serum Proteins

Design: Neutral oligonucleotide (e.g., morpholino) on the outside, charged oligonucleotide (e.g., phosphorothioate) on the inside.

One reason why oligonucleotides have not gained a major foothold in the clinic is that several are associated with severe side effects that are triggered, at least in part, by their charge-based association with serum proteins. For this reason, charge-neutral oligonucleotides, like morpholinos, have become popular, but their design inhibits protein interactions that may be desired, such as the interaction of oligonucleotides with toll-like receptors. The biradial SNA design would allow for the incorporation of a charged nucleic acid species (such as a phosphorothioate backbone oligonucleotide) on the inside, where it is shielded from potentially adverse interactions with serum proteins, and a more benign uncharged nucleic acid species (such as a morpholino oligonucleotide) can be positioned on the outside. With this design, it is expected that there would be better tolerance of a particular dose of oligonucleotide therapy.

### Methods and Experiments

Hetero-biradial SNAs will be produced as described herein (e.g., Example 1) with a dummy oligonucleotide sequence (e.g., T20) positioned on the inside and outside. The inner oligonucleotide sequence will possess an anionic phosphorothioate oligonucleotide (5′-Chol-sp18-Cy5-sp18-TTT TTT TTT TTT TTT TTT TT-3′, (SEQ ID NO: 8), phosphorothioate (PS) backbone). The outer oligonucleotide will possess a charge-neutral morpholino backbone (5′-Chol-sp18-Cy3-sp18-TTT TTT TTT TTT TTT TTT TT-3′, (SEQ ID NO: 9), morpholino (PMO) backbone). Control SNAs will include a hetero-biradial SNA with the position of the oligonucleotides swapped (PS on the outside, PMO on the inside) and a conventional SNA with the PS-backbone oligonucleotide on the outside. These oligonucleotides will be intravenously administered to C57BL/6 or BALB/c mice every other day for 4 weeks and, at the end of the study period, the biodistribution of the PS and PMO oligonucleotides will be analyzed, along with measures of liver toxicity such as serum transaminase levels and systemic inflammation such as C-reactive protein. It is expected that the biodistribution of the phosphorothioate oligonucleotide will be distinct when it is on the inside of a hetero-biradial SNA compared to if it is displayed on the outside owing to decreased interaction with serum proteins, which will lead to lower liver accumulation and toxicity as well as lower systemic inflammation.

1. A biradial spherical nucleic acid (SNA) comprising:
(a) a liposomal core comprising an outer leaflet and an inner leaflet; and
(b) a population of oligonucleotides attached to the liposomal core, the population of oligonucleotides comprising:
(i) a first plurality of oligonucleotides attached to the outer leaflet of the liposomal core and radiating outward from the liposomal core; and
(ii) a second plurality of oligonucleotides attached to the inner leaflet of the liposomal core and radiating inward into the liposomal core, wherein one or more or all of the second plurality of oligonucleotides is a nuclease resistant oligonucleotide, and
wherein the first plurality of oligonucleotides and the second plurality of oligonucleotides are different.

2. The biradial SNA of claim 1, wherein the liposomal core is comprised of one or more high melting temperature lipids.

3. A biradial spherical nucleic acid (SNA) comprising:
(a) a liposomal core comprising an outer leaflet and an inner leaflet, wherein the liposomal core is comprised of one or more high melting temperature lipids; and
(b) a population of oligonucleotides attached to the liposomal core, the population of oligonucleotides comprising:
(i) a first plurality of oligonucleotides attached to the outer leaflet of the liposomal core and radiating outward from the liposomal core; and
(ii) a second plurality of oligonucleotides attached to the inner leaflet of the liposomal core and radiating inward into the liposomal core, and wherein the first plurality of oligonucleotides and the second plurality of oligonucleotides are different.

4. The biradial SNA of claim 1, wherein the first plurality of oligonucleotides has a surface density of about 0.5 pmol/cm² to about 2 pmol/cm², and/or the second plurality of oligonucleotides has a surface density of about 0.3 pmol/cm² to about 1.5 pmol/cm².

5. (canceled)

6. (canceled)

7. (canceled)

8. The biradial SNA of claim 1, wherein the liposomal core has a diameter of about 30 nanometers (nm) to about 100 nm.

9. (canceled)

10. The biradial SNA of claim 1, wherein the SNA has a diameter of about 30 nanometers (nm) to about 100 nm.

11. The biradial SNA of claim 2, wherein the one or more high melting temperature lipids is a saturated phosphatidylcholine phospholipid comprising a fatty acid tail length between 16 and 24, a saturated phosphatidylethanolamine lipid with a fatty acid tail length between 16 and 24, a saturated phosphatidylglycerol phospholipid with a fatty acid tail length between 16 and 24, a saturated phosphatidic acid phospholipid with a fatty acid tail length between 16 and 24, a cardiolipin-based phospholipid with a fatty acid tail length between 14 and 16, or a combination thereof.

12. The biradial SNA of claim 1, wherein the liposomal core is comprised of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), or cholesterol.

13. (canceled)

14. (canceled)

15. (canceled)

16. The biradial SNA of claim 1, wherein one or more or all of the first plurality of oligonucleotides is a nuclease resistant oligonucleotide.

17. The biradial SNA of claim 3, wherein one or more or all of the second plurality of oligonucleotides is a nuclease resistant oligonucleotide.

18. The biradial SNA of claim 1, wherein the nuclease resistant oligonucleotide comprises a chemical modification.

19. The biradial SNA of claim 1, wherein one or more oligonucleotides in the population of oligonucleotides comprises a toll-like receptor (TLR) agonist.

20. (canceled)

21. (canceled)

22. The biradial SNA of claim 19, wherein the TLR agonist is a toll-like receptor 3 (TLR-3) agonist, toll-like receptor 7 (TLR-7) agonist, toll-like receptor 9 (TLR-9) agonist, or a combination thereof.

23. The biradial SNA of claim 1, wherein one or more or all oligonucleotides in the first plurality of oligonucleotides is an aptamer.

24. A method of producing a biradial spherical nucleic acid (SNA), the biradial SNA comprising a liposomal core and a population of oligonucleotides attached to the liposomal core, the method comprising:
(a) creating a mixture by contacting the population of oligonucleotides with a population of phospholipids, wherein one or more oligonucleotides in the population of oligonucleotides comprises a hydrophobic anchor group, and wherein the contacting occurs in an aqueous buffer;
(b) subjecting the mixture to one or more freeze-thaw cycles; and
(c) subjecting the mixture to a process by which diameter of the biradial SNA is reduced,
wherein steps (a)-(c) result in a first plurality of oligonucleotides from the population of oligonucleotides being attached to the outer leaflet of the liposomal core and radiating outward from the liposomal core; and a second plurality of oligonucleotides from the population of oligonucleotides being attached to the inner leaflet of the liposomal core and radiating inward into the liposomal core,
thereby producing the biradial SNA.

25. The method of claim 24, wherein the process is a sonication process, or the process is an extrusion process.

26. (canceled)

27. (canceled)

28. (canceled)

29. (canceled)

30. (canceled)

31. The method of claim 24, further comprising placing the biradial SNA in a matrix and subjecting the biradial SNA to an electric field, thereby extracting the first plurality of oligonucleotides from the outer leaflet resulting in a liposome comprising the second plurality of oligonucleotides attached to the inner leaflet of the liposomal core.

32. (canceled)

33. (canceled)

34. (canceled)

35. The method of claim 24, wherein one or more oligonucleotides in the population of oligonucleotides comprises a toll-like receptor (TLR) agonist.

36. (canceled)

37. (canceled)

38. (canceled)

39. (canceled)

40. (canceled)

41. (canceled)

42. (canceled)

43. A method of treating a disorder comprising administering an effective amount of the biradial SNA of claim 1 to a subject in need thereof, wherein the administering treats the disorder.

44. The biradial SNA of claim 3, wherein the one or more high melting temperature lipids is a saturated phosphatidylcholine phospholipid comprising a fatty acid tail length between 16 and 24, a saturated phosphatidylethanolamine lipid with a fatty acid tail length between 16 and 24, a saturated phosphatidylglycerol phospholipid with a fatty acid tail length between 16 and 24, a saturated phosphatidic acid phospholipid with a fatty acid tail length between 16 and 24, a cardiolipin-based phospholipid with a fatty acid tail length between 14 and 16, or a combination thereof.


SEQUENCE LISTING (9 sequences)

SEQ ID NO: 1 - DNA, 20 bp, synthetic construct
  source 1..20: mol_type = other DNA
  misc_binding 1: bound_moiety = Cholesterol spacer18 Cy5 spacer18
tccatgacgttcctgacgtt

SEQ ID NO: 2 - DNA, 20 bp, synthetic construct
  source 1..20: mol_type = other DNA
  misc_binding 1: bound_moiety = Cholesterol spacer18 Cy5 spacer18
tttttttttttttttttttt

SEQ ID NO: 3 - DNA, 19 bp, synthetic construct
  source 1..19: mol_type = other DNA
  misc_binding 1: bound_moiety = Cholesterol spacer18 Cy5 spacer18
tcccagtgcccagtccaac

SEQ ID NO: 4 - DNA, 24 bp, synthetic construct
  source 1..24: mol_type = other DNA
  misc_binding 1: bound_moiety = Cholesterol spacer18 Cy3 spacer18
cctggatgggaacttaccgctgca

SEQ ID NO: 5 - DNA, 20 bp, synthetic construct
  source 1..20: mol_type = other DNA
  misc_binding 1: bound_moiety = Cholesterol spacer18 Cy3 spacer18
ccttccctgaaggttcctcc

SEQ ID NO: 6 - DNA, 19 bp, synthetic construct
  source 1..19: mol_type = other DNA
  misc_binding 1: bound_moiety = Cholesterol spacer18 Cy3 spacer18
gcgtggtaccacgcttttt

SEQ ID NO: 7 - DNA, 21 bp, synthetic construct
  source 1..21: mol_type = other DNA
  misc_binding 1: bound_moiety = Cholesterol sp18 Cy3 sp18
ggtggtggtggtggtggtggt

SEQ ID NO: 8 - DNA, 20 bp, synthetic construct
  source 1..20: mol_type = other DNA
  misc_binding 1: bound_moiety = Chol sp18 Cy5 sp18
tttttttttttttttttttt

SEQ ID NO: 9 - DNA, 20 bp, synthetic construct
  source 1..20: mol_type = other DNA
  misc_binding 1: bound_moiety = Chol sp18 Cy3 sp18
tttttttttttttttttttt